(19) United States
(12) Patent Application Publication
(10) Pub. No.: US 2008/0262254 A1
(43) Pub. Date: Oct. 23, 2008

(54) Sphingolipids

(76) Inventors: Peter Ettmayer, Wien (AT); Klemens Hogenauer, Wien (AT); Peter Nussbaumer, Wien (AT); Carsten Peters, Wien (AT); Klaus Weigand, Wien (AT); Michael Ghobrial, Wien (AT); Thomas Ullrich, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/915,683

(22) PCT Filed: May 29, 2006
(86) PCT No.: PCT/EP2006/005105

§ 371 Date: Jul. 2, 2008

(30) Foreign Application Priority Data
May 31, 2005 (GB) 0511062.2
Jun. 10, 2005 (GB) 0511845.0
Jun. 16, 2005 (GB) 0512322.9

(51) Int. Cl. C07C 233/18

(52) U.S. Cl. 554/36

(57) ABSTRACT

The use of a metathesis catalyst in the preparation of a functionalized sphingolipid.

The present invention relates to organic compounds, such as a sphingolipid.

The biological function of sphingolipids is not merely structural, but most of these molecules act as extra- and intracellular signalling mediators, see e.g. B. J. Pettus et al, Curr. Mol. Medicine, 2004, 4, 405. Bioactive sphingolipids with an attached label or tag are useful tools for localisation, binding and metabolism studies and as assay substrates.

A sphingolipid is an aminoalcohol comprising at least one hydrocarbon chain. Sphingolipids as used herein include ceramides, sphingosines and sphingomyelins and where appropriate their corresponding phosphate, glyco and other conjugates.

We have now surprisingly found a process for the production of functionalised sphingolipids in one step, wherein the group comprising the functional group which is introduced by that process is attached to a carbon atom of the hydrocarbon chain of the corresponding sphingolipid, starting from a sphinglipid wherein the carbon atom to which the functional group is to be attached is substituted by hydrogen or alkyl, preferably hydrogen.

In one aspect the present invention provides a process for the production of a functionalized sphingolipid wherein
its hydrocarbon chain comprises a carbon-carbon double bond,
its hydrocarbon chain is functionalized, and, optionally, e.g. preferably,
the amine group which is part of its aminoalcohol is substituted, e.g. protected, comprising reacting a sphingolipid, wherein
the hydrocarbon chain of said sphingolipid comprises a carbon-carbon double bond, and, optionally
the amine group which is part of its aminoalcohol is substituted, e.g. protected, with a functionalizing reactant comprising a carbon-carbon double bond, in the presence of a metathesis catalyst, e.g. a Grubbs' second generation catalyst, and in the presence of organic solvent, and
   isolating a functionalized sphingolipid obtained from the reaction mixture.

According to the present invention it was found that the only requirements in a process according to the present invention are
that there is a carbon-carbon double bond, preferably one, in the hydrocarbon chain of the sphingolipid used as a starting material
that there is a carbon-carbon double bond, preferably one at the N-terminus, in the reactant comprising the functionalizing group,
that the sphingolipid and the reactant are of such chemical nature, that a functionalized sphingolopid is obtained which is different from the sphingolipid used as a starting material, and,
that groups in a sphingolipid or a functionalizing group which are reactive under or not compatible with the reaction conditions are protected.

A hydrocarbon chain as used herein comprises an alkenyl chain having 3 to 24 carbon atoms, preferably 6 to 24 carbon atoms, such as 6 to 18 carbon atoms, and one carbon-carbon double bond in any position of the hydrocarbon chain. A hydrocarbon chain optionally may be substituted, e.g. by substituents as usual in sphingolipid chemistry.

According to the present invention a hydrocarbon chain in a sphingolipid may be attached to the amine group of the aminoalcohol, e.g. via a —CO— or O—CO— group, or to the carbon atom carrying the hydroxy group of the aminoalcohol and is preferably attached to the carbon atom carrying the hydroxy group of the aminoalcohol.

In both, in a sphingolipid used as a starting material and in a functionalizing reactant according to the present invention, there is preferably one single carbon-carbon double bond present.

According to the present invention the carbon-carbon double bond may be in any position of the hydrocarbon chain. A preferred position includes that position in the sphingolipid which is adjacent to the carbon atom which carries the hydroxy group of the aminoalcohol.

A “functionalized sphingolipid” as used herein means a sphingolipid which is different from the sphingolipid used as a starting material in a process according to the present invention. The difference of a functionalized sphingolipid compared with a sphingolipid used as a starting material may be a difference in chain length, and/or a different substitution pattern.

A functionalized sphingolipid as used herein comprises preferably as a difference compared to the sphingolipid used as a starting material
a chemical reactive function which is preferably in protected form,
a leaving group, or
a group which is detectable by physical means, optionally in protected form, preferably a group detectable by physical means. Such groups may be comprised in a functionalizing group beside groups which themselves are not reactive, e.g. and which may function as linkers.

A functionalizing reactant as used herein comprises a hydrocarbon chain of 3 to 22 carbon atoms comprising one carbon-carbon double bond and optionally comprising
a group which is detectable by physical means, optionally in protected form, or
a leaving group, and/or
a chemical reactive function which is preferably in protected form, and
optionally one or more linkers;
optionally a different chain length.

Protecting groups which protect a reactive chemical group in a functionalizing group, leaving groups which are prone to easy replacement by substitution and groups detectable by physical means, optionally in protected form are known or may be provided as appropriate, e.g. according, e.g. analogously, to a process as conventional.

Suitable protecting groups which protect a reactive chemical group are dependent on the nature of the reactive function.

Suitable leaving groups are groups which may be conveniently replaced by another group, e.g. substituted by a group which comprises a group which is detectable by physical means, such as a group comprising a group which is detectable by physical means attached to a linker group.

Suitable linker groups are known and may be provided as appropriate, e.g. according, e.g. analogously to a process as conventional.

A group which is detectable by physical means includes a labeling group, which is, e.g. selectively, detectable by physical means.

Suitable labeling groups which are detectable by physical means include e.g.
groups selectively detectable by fluorimetric means, such as a group labeled with a fluorescent group, e.g. a group originating from a fluorescent dye, e.g. including pyrenes, dansyls (compounds that contain a 1-dimethylaminonaphthalene-5-sulfonyl group), nitrobenzo-2-oxa-1,3-diazols (NBDs), fluorenes and fluorenones; and fluorescent BODIPY dyes, e.g. such as provided by Invitrogen, preferably a nitrobenzo-2-oxa-1,3-diazol, a fluorene or a BODIPY dye,
groups selectively detectable by selective photoactivation, such as a group useful as photoaffinity probe, e.g. a benzophenone; or
groups selectively detectable by bioaffinity methods, such as a group useful for bioaffinity-tagging, e.g. a biotin, such as biotin.

Preferred labeling groups include e.g groups of formulae

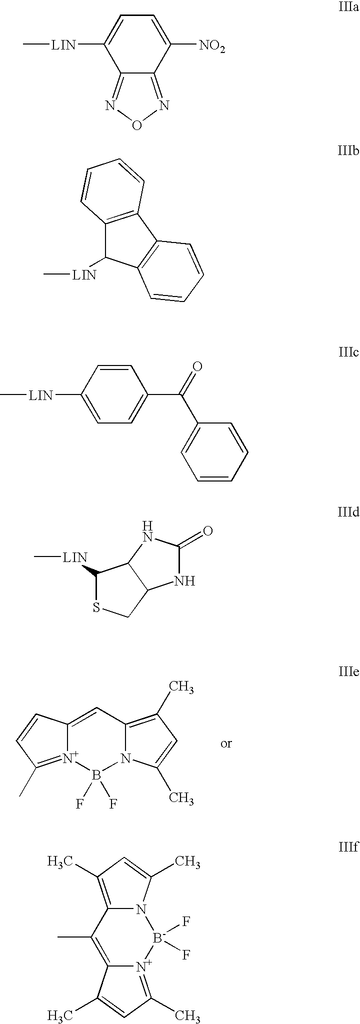

wherein LIN is a linker,
e.g. LIN includes in case of a group of
formula IIIa the —NH— group,
formula IIb the —NH—CO—O—CH₂— group,
formula IIIc the —O— group, e.g. or no linker,
formula IIId the —NH—CO—(CH₂)₃—CH₂— group, and
formulae IIIe and IIIf there is no linker group.

The amine group of the aminoalcohol in the sphingolipid is optionally substituted. Suitable amino substituents include substituents such as appropriate, preferably
an acyl group, e.g. (C₂₋₂₅)acyl, such as —CO—(C₁₋₂₄)alkyl or a —CO—(C₂₋₂₄)alkylene; such as a —CO-hydrocarbon chain,
an alkyl- or akenyloxycarbonyl group, such as —CO—O—(C₁₋₂₄)alkyl, such as —CO—O—(C₁₋₆)alkyl, e.g. tert-butoxycarbonyl or —CO—O—(C₂₋₂₄)alkylene, e.g. a —CO—O-hydrocarbon chain, or
one or two alkyl groups, e.g. one or two (C₁₋₂₄)alkyl, such as (C₁₋₄)alkyl, e.g. two (C₁₋₄)alkyl.

Sphingolipids useful as a starting material in a process according to the present invention include a compound of formula

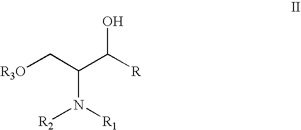

such as a compound of formula

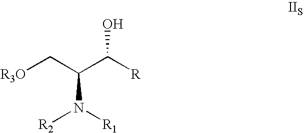

wherein
R is (C₁₋₂₄)alkyl or a hydrocarbon chain, such as (C₃₋₂₄)alkylene,
R₁ and R₂ independently of each other are
—H,
alkyl, such as (C₁₋₂₄)alkyl, e.g. (C₁₋₄)alkyl,
a hydrocarbon chain, such as (C₃₋₂₄)alkylene,
acyl, such as (C₂₋₂₅)acyl, e.g. a group of formula —CO—R′, wherein
   —R′ is (C₁₋₂₄)alkyl or a hydrocarbon chain, such as (C₃₋₂₄)alkylene, or
   R′ is alkoxy, e.g. (C₁₋₆)alkoxy, or alkenyloxy, e.g. comprising a hydrocarbon chain, e.g. (C₃₋₂₄)alkenyloxy,
      R₃ is H or a phosphor containing group, e.g. (HO)₂PO, e.g. which phosphor containing group is optionally alkylated, e.g. by (C₁₋₄)alkyl, such as aminoalkyl, e.g. R₃ is phosphorylcholinyl of formula

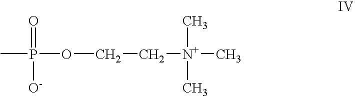

with the proviso that
if R₁ is acyl, than R₂ is H,
at least one hydrocarbon chain comprising a carbon-carbon double bond is present in a compound of formula II,
preferably one single carbon-carbon double bond is present in a compound of formula II;
   preferably
—R₁ is H or (C₁₋₄)alkyl;
   preferably
   R₂ is H or (C₁₋₂₄)alkylcarbonyl, such as (C₁₋₁₈)alkylcarbonyl, or (C₁₋₈)alkoxycarbonyl, such as tert.butoxycarbonyl.

Functionalized sphingolipids according to the present invention include a compound of formula

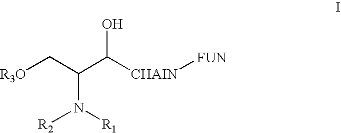

such as a compound of formula

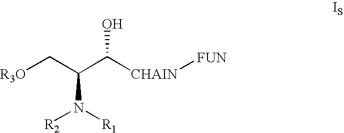

wherein R₁, R₂ and R₃ are as defined above, CHAIN is a hydrocarbon chain comprising one double bond and comprising 3 to 24 carbon atoms in total, and
FUN is a group comprising a detectable group, a leaving group and/or a reactive chemical group optionally together with appropriate linker groups, such as a detectable group, optionally beside a linker group.

In another aspect the present invention provides a process according to the present invention, e.g. wherein the functionalized sphingolipid is a compound of formula I, wherein the residues are as defined above, and which is different from a functionalized sphingolipid according to the present invention, which process comprises

reacting a sphingolipid wherein the hydrocarbon chain of said sphingolipid comprises a carbon-carbon double bond, and, optionally, the amine group which is part of its aminoalcohol is substituted, such as a compound of formula I, wherein the residues are as defined above,
with a compound of formula

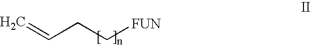

e.g. which is a funtionalizing reactant, wherein
n is a number which is dependent from the desired chain length of the desired functionalized hydrocarbon chain, and
FUN is functionalizing group, e.g. a group comprising at least one
detectable group, or
   leaving group, and/or
reactive chemical group in protected form,
e.g. beside one or more linker groups,
   preferably a leaving group optionally beside linker groups, in the presence of a metathesis catalyst, e.g. a Grubbs' catalyst, preferably a second generation Grubbs' catalyst (Grubbs' II catalyst),
   e.g. in the presence of an organic solvent, such as a polar organic solvent, e.g. a halogenated hydrocarbon such as CH₂Cl₂, optionally in the presence of a cosolvent, e.g. N,N-dimethylformamide (DMF), or dimethylsulfoxide (DMSO),
   and isolating the functionalized sphingolipid obtained from the reaction mixture.

In a group FUN, at least one detectable group, a leaving group or a reactive chemical group is present, e.g. a detectable group, or a leaving group and/or a reactive chemical group in protected form may be present optionally bound via linkers, e.g. one or more or none linkers. Suitable linkers are linkers as appropriate, e.g. including linkers obtained according, e.g. analogously, to a process as conventional, e.g. including alkylene of a desired length, e.g. (C₁₋₆)alkylene, and optionally a functional chemical linker group, such as an oxygen, an amine group, an amide group, an ester group, a carbamoyl group or the like.

n in a compound of formula II is dependent on the desired chain length of the hydrocarbon chain in a desired functionalized sphingolipid obtainable according to the present invention. n is the desired chain length in a functionalized sphingolipid according to the present invention minus 3, counted from and including the C—C double bond.

E.g. in a compound of formula

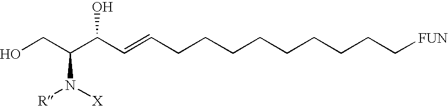

the desired chain length counted from and including the C—C double bond is 11 and n in a compound of formula II is 8.

E.g. in a compound of formula

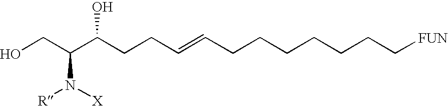

the desired chain length counted from and including the C—C double bond is 9 and n in a compound of formula II is 6.

The production of compounds of formula II is known or may be carried out as appropriate, e.g. according, e.g. analogously, to a method as conventional.

E.g. if FUN in a compound of formula I is a group of formula

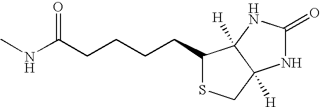

a corresponding functionalizing reactant of formula II may be prepared according to the following reaction scheme:

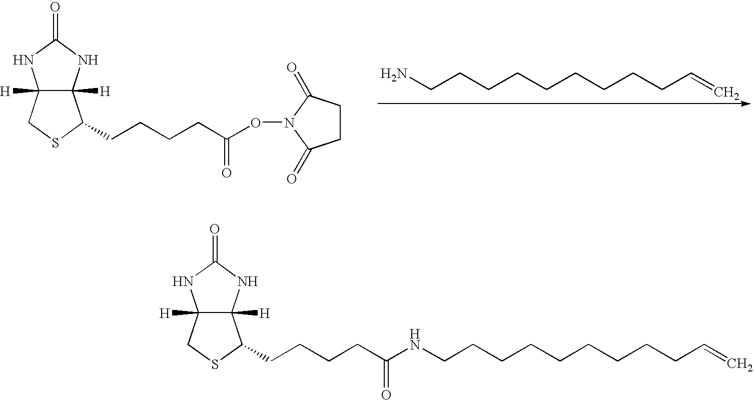

Metathesis catalysts, e.g. Grubbs' catalysts, are known, see e.g. T. M. Trnka and R. H. Grubbs, Acc. Chem. Res., 2001, 34, 18; A. Fürstner, Angew. Chem. int. Ed., 2000, 39, 3012; S. J. Connon and S. Blechert, Angew. Chem. Int Ed., 2003, 42, 1900; M. Scholl, S. Ding, C. W. Lee, R. H. Grubbs, Org. Lett., 1999, 1, 953, or may be provided by a method as appropriate, e.g. by a method as conventional.

A process according to the present invention may be carried out as follows.

A sphingolipid, wherein the hydrocarbon chain of said sphingolipid comprises a carbon-carbon double bond, and, optionally the amine group which is part of its aminoalcohol is substituted, e.g. protected and a functionalizing reactant according to the present invention comprising a carbon carbon double bond are mixed with organic solvent, e.g. dissolved in organic solvent, optionally in the presence of a co-solvent. To the mixture obtained a metathesis catalyst is added and the reaction mixture is heated under stirring to temperatures up to the reflux temperature of the solvent (system), e.g. for several hours. A functionalized sphingolipid according to the present invention is obtained, is isolated from the reaction mixture and further purified by a method as appropriate, e.g. according, e.g. analogously to a method as conventional, such as chromatography.

For one equivalent of the sphingolipid used as the starting material,
at least one, or more, preferably 2 to 5, such as 3 to 4 equivalents of a the functionalizing reactant may be conveniently used,
a catalytic amount of the catalyst, preferably in a range of 0.01 to 0.5 equivalents, e.g. 0.05 to 0.2, may be conveniently used.

A functionalized sphingolipid obtained according to a process of the present invention may be further reacted to obtain another sphingolipid, such as a functionalized sphingolipid according to the present invention. Further reaction e.g. includes
salt formation,
saturation of the C—C double bond to obtain an alkyl chain, such as a hydrocarbon chain as defined herein, but which is saturated,
in case that a functionalizing group comprises a protected reactive function deprotection may follow,
in case that a functionalizing group comprises a leaving group, such leaving group may be replaced by another desired group, e.g. via a substitution reaction, e.g. a leaving group may be replaced by a group detectable by physical means.

Such further reactions may be carried out as appropriate, e.g. according, e.g. analogously, to a process as conventional.

In another aspect the present invention provides a process according to the present invention to obtain a functionalized sphingolipid according to the present invention and further reacting to obtain another sphingolipid, e.g. another functionalized sphingolipid according to the present invention.

In another aspect the present invention provides a process for the production of a functionalized sphingolipid according to the present invention, wherein the amine group of the aminoalcohol is unsubstituted, comprising
a) reacting a sphingolipid wherein the amine group of the aminoalcohol is protected, e.g. protected by a group which may be removed under appropriate pH conditions, such as a —CO—O-alkyl group, e.g. —CO—O—(C₁₋₈)alkyl; such as. tert-butoxycarbonyl according to a process of the present invention,
b) splitting off the amine protecting group, and
d) isolating the functionalized sphingolipid wherein the amine group is unsubstituted from the reaction mixture.

Deprotection in step b) may be carried under appropriate pH conditions. E.g. if a —CO—O-alkyl group has been used for amine protection, such group may be split off under a acidic conditions, e.g. by use of HCl, HCl in organic solvent, or TFA, e.g. according, e.g. analogously, to a method as conventional.

The process according to the present invention is also useful for the preparation of functionalized sphingolipids such as disclosed and claimed in WO2005030780. Functionalized sphingolipids as disclosed herein or in WO2005030780 wherein the amino group is unsubstituted may be also obtained by producing a functionalized sphingolipid according, e.g. analogously, to a process of the present invention wherein the amine group is protected, followed by deprotection, e.g. using a Boc protecting group as in Example 5 of the present invention as a protecting group, and deprotecting.

In another aspect the present invention provides a compound selected from the group consisting of compounds of formula

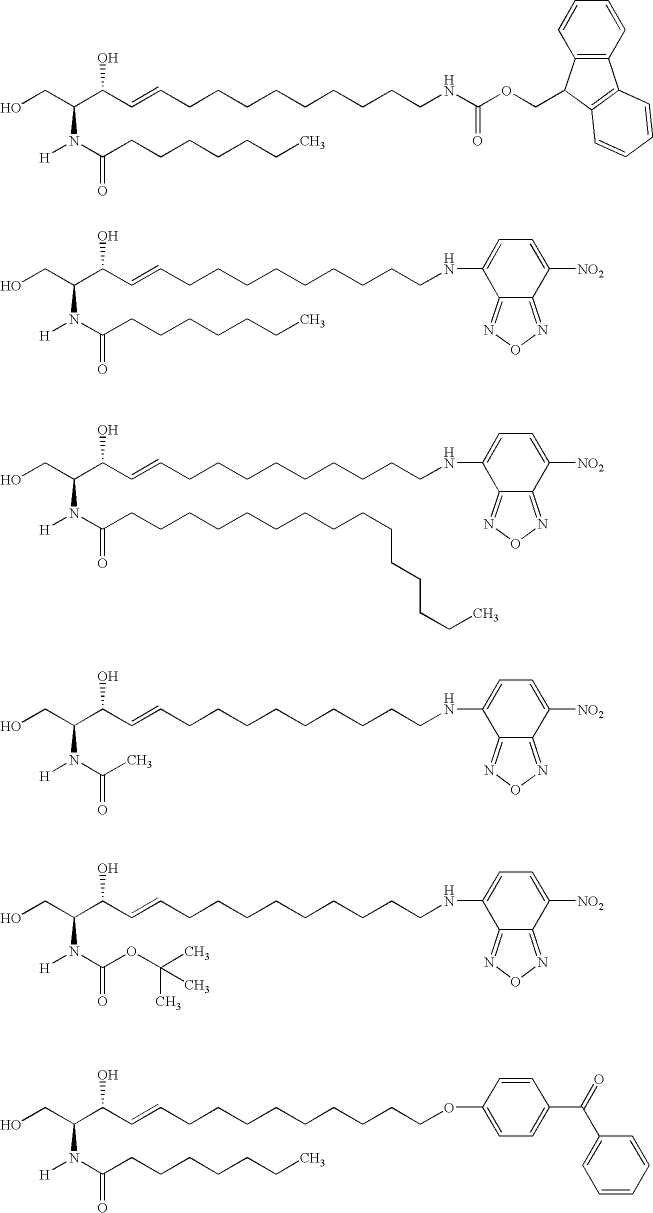

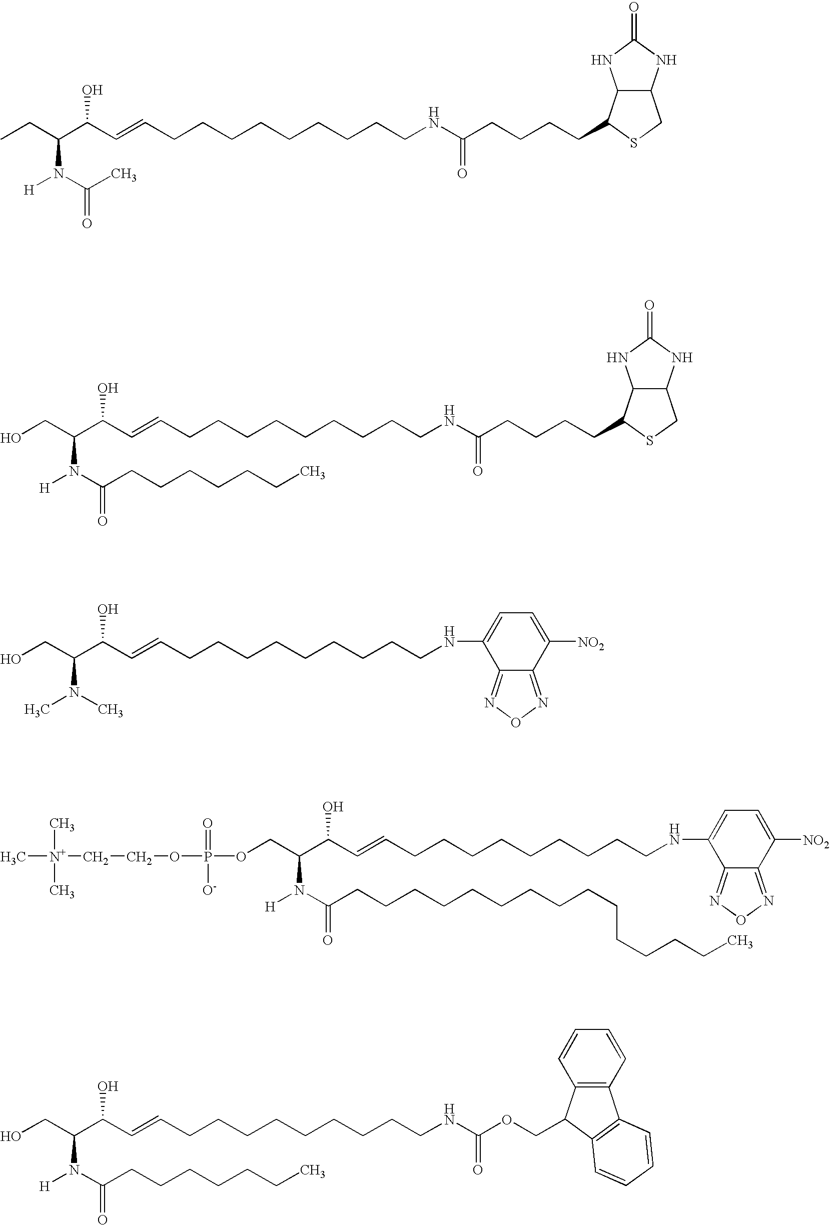

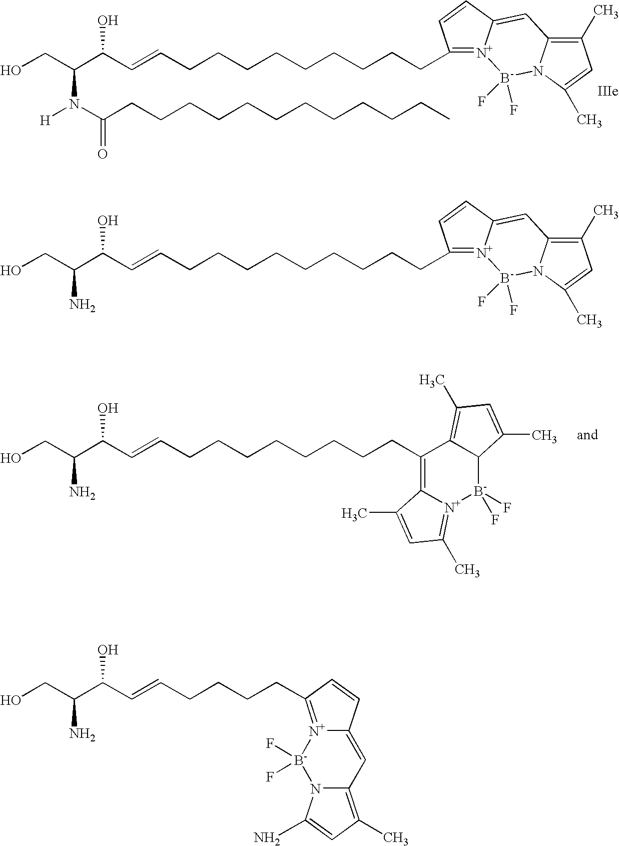

e.g. which are compounds of Examples 1 to 15.

A compound of any of the Examples 1 to 15 is herein also designated as “compound(s) of (according to) the present invention”. A compound of the present invention includes a compound in any form, e.g. in free form, in the form of a salt, in the form of a solvate and in the form of a salt and a solvate.

In another aspect the present invention provides a compound of any of the Examples 1 to 15 in the form of a salt.

A salt of a compound of the present invention e.g. includes acid addition salts, with inorganic and organic acids, e.g. a salt of a compound of formula I with hydrochloric acid, hydrobromic acid, sulphuric acid, trifluoroacetic acid, hydrogen fumaric acid, fumaric acid, naphthalin-1,5-sulphonic acid, preferably hydrochloric acid or trifluoroacetic acid.

A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention may exist in the form of isomers and mixtures thereof; e.g. optical isomers, diastereoisomers, cis/trans conformers. A compound of the present invention may e.g. contain asymmetric carbon atoms and may thus exist in the form of enantiomers or diastereoisomers and mixtures thereof, e.g. racemates. A compound of the present invention may be present in the (R)-, (S)- or (R,S)-configuration regarding specific substituents, preferably in the (R)- or (S)-configuration. For example, in a compound of formula I the carbon atom carrying the hydroxy group and the carbon atom carrying the amine group of the aminoalcohol, both are asymmetric carbon atoms and a compound of the present invention may be correspondingly in the R- and S-configuration, including mixtures thereof, regarding substituents at these asymmetric carbon atoms. E.g. a compound of formula I also comprises a carbon-carbon double bond and substituents attached to said double bond may be in the form of cis/trans isomers.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of formula I, where tautomers can exist.

In another aspect the present invention provides the use of a sphingolipid, functionalized according to a process of the present invention, as a biological tool, such as an assay substrate and/or a visualization reagent.

In another aspect the present invention provides the use of a metathesis catalyst in the preparation of a functionalized sphingolipid.

In another aspect the present invention provides a one-step process for the production of a functionalized sphingolipid
which is functionalized at the hydrocarbon chain of the corresponding sphingolipid,
which hydrocarbon chain comprises one C—C double bond, and
wherein its amine group is optionally substituted,
   starting from a sphingolipid, wherein its amine group is optionally substituted and wherein its hydrocarbon chain comprises one C—C double bond.

### EXAMPLE 1

### Production of a Functionalised Sphingolipid of Formula

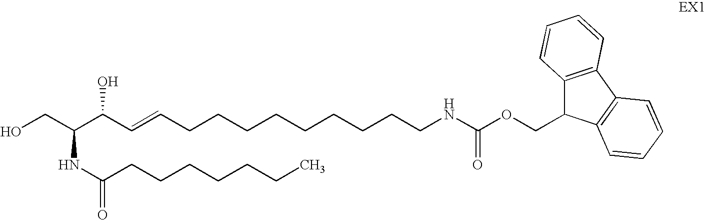

A sphingolipid of formula

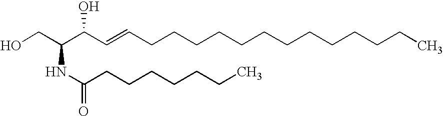

is dissolved in CH₂Cl₂ and the mixture obtained is treated with 3 to 4 equivalents of a compound of formula

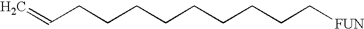

and 0.1 equivalents of 2^{nd} generation Grubbs' catalyst The mixture obtained is heated to reflux for 2.5 to 4 hours. From the mixture obtained solvent is evaporated, and the evaporation residue is subjected to column chromatography on sephadex LH20 followed by preparative PLC. A compound of formula EX1 is obtained. Yield: 49% of theory.

Analogously as described in Example 1, but using appropriate starting materials and adding in case of C₁₆-ceramide, sphingomyelin or biotinylated undecenyl a few drops of N,N-dimethylformamide to CH₂Cl₂ as a co-solvent, compounds of formula

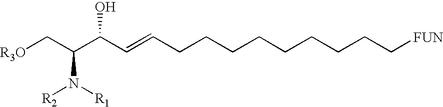

### in Examples 1 to 13 of TABLE 1,

a compound of formula

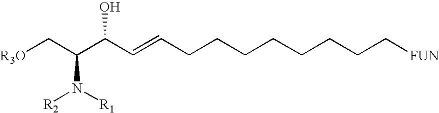

### in Examples 14 of the TABLE, and

a compound of formula

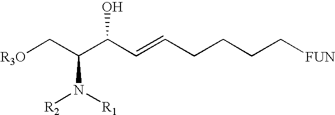

### in Example 15 of the TABLE,

are obtained wherein
FUN is a group of the formula as indicated in TABLE 1, which are groups of formulae

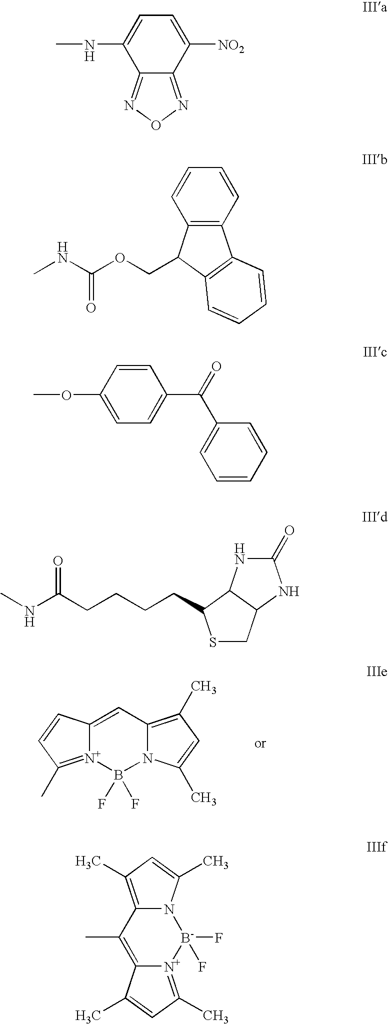

and
R₁, R₂ and R₃ are as defined in TABLE 1 below, and
R₃ is H in Examples 1 to 9 and 11 to 15 and phosphorylcholinyl of formula IV in Example 10. Yields (not optimised) are also indicated in TABLE 1. “EX” means “Example number”.

### TABLE 1

TABLE 1

| EX | R₁ | R₂ | FUN | R₃ | Yield^{a} |
|---|---|---|---|---|---|
| 2 | —CO—C₇H₁₅ | H | IIIa | H | 52% |
| 3 | —CO—C₁₅H₃₁ | H | IIIa | H | 52% |
| 4 | —CO—CH₃ | H | IIIa | H | 48% |
| 5 | —CO—OC(CH₃)₃ | H | IIIa | H | 71% |
| 6 | —CO—C₇H₁₅ | H | IIIc | H | 20% |
| 7 | —CO—CH₃ | H | IIId | H | 22% |
| 8 | —CO—C₇H₁₅ | H | IIId | H | 14% |
| 9*) | CH₃ | CH₃ | IIIa | H | 39% |
| 10 | —CO—C₁₅H₃₁ | H | IIId | VI | 5% |
| 11 | —CO—C₇H₁₅ | H | IIIb | H | 49% |
| 12 | —CO—C₁₅H₃₁ | H | IIIe | H | 48% |
| 13 | H | H | IIIe | H | 31% |
| 14 | H | H | IIIf | H | 26% |
| 15 | H | H | IIIe | H | 32% |

*)Obtained in the form of a hydrochloride.

*) Obtained in the form of hydrochloride.

Compounds of Examples 13 to 15 are obtained starting from a corresponding sphingolipid wherein the amine group of the aminoalcohol is N-tert-butoxycarbonyl-protected, obtaining a Functionalized sphingolipid of the present invention wherein the amine group of the aminoalcohol is N-tert-butoxycarbonyl-protected and splitting off the protecting group by use of HCl in dioxane.

1. A process for the production of a functionalized sphingolipid comprising a hydrocarbon chemical and an aminoalcohol group wherein
its hydrocarbon chain comprises a carbon-carbon double bond,
its hydrocarbon chain is functionalized, and, optionally,
the amine group which is part of its aminoalcohol is substituted,
the process comprising the step of reacting a sphingolipid, wherein
the hydrocarbon chain of said sphingolipid comprises a carbon-carbon double bond,
and, optionally
the amine group which is part of its aminoalcohol is substituted,
with a functionalizing reactant comprising a carbon-carbon double bond, in the presence of a metathesis catalyst, and
isolating a functionalized sphingolipid obtained from the reaction mixture.

2. A process according to claim 1, wherein a sphingolipid, wherein
the hydrocarbon chain of said sphingolipid comprises a carbon-carbon double bond,
and, optionally
the amine group which is part of its aminoalcohol is substituted is a compound of formula 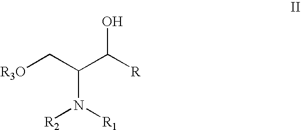
wherein
R is (C₁₋₂₄)alkyl or (C₃₋₂₄)alkylene,
R₁ and R₂ independently of each other are H, (C₁₋₂₄)alkyl, (C₃₋₂₄)alkylene, or (C₂₋₂₅)acyl, and
R₃ is H or a phosphorus containing group.

3. A process according to claim 1, wherein a functionalizing reactant is a compound of formula 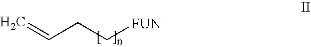
wherein
n is a number which is dependent from the desired chain length of the desired functionalized hydrocarbon chain, and
FUN is a group comprising at least one
detectable group, or
leaving group, and/or
reactive chemical group in protected form,
optionally beside one or more linker groups,

4. A process according to claim 1, wherein a functionalized sphingolipid as defined in claim 1, further comprises
a group which is detectable by physical means, optionally in protected form, or
a leaving group, and/or
a chemical reactive function in protected form, and
optionally one or more linker groups.

5. A process according to claim 4, wherein a group which is detectable by physical means is a labeling group which are selected from a group selectively detectable
by fluorimetric means,
by selective photoactivation, or
by a bioaffinity method.

6. A process according to claim 5, wherein a labeling group is a group of formula 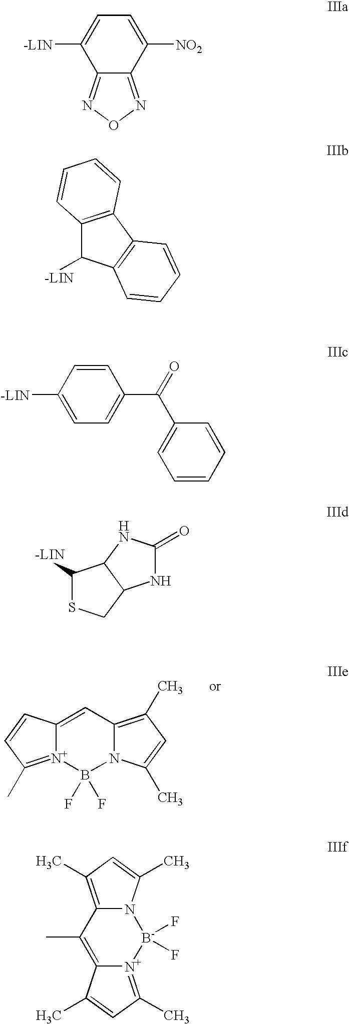
wherein LIN is a linker group.

7. A compound selected from the group consisting of

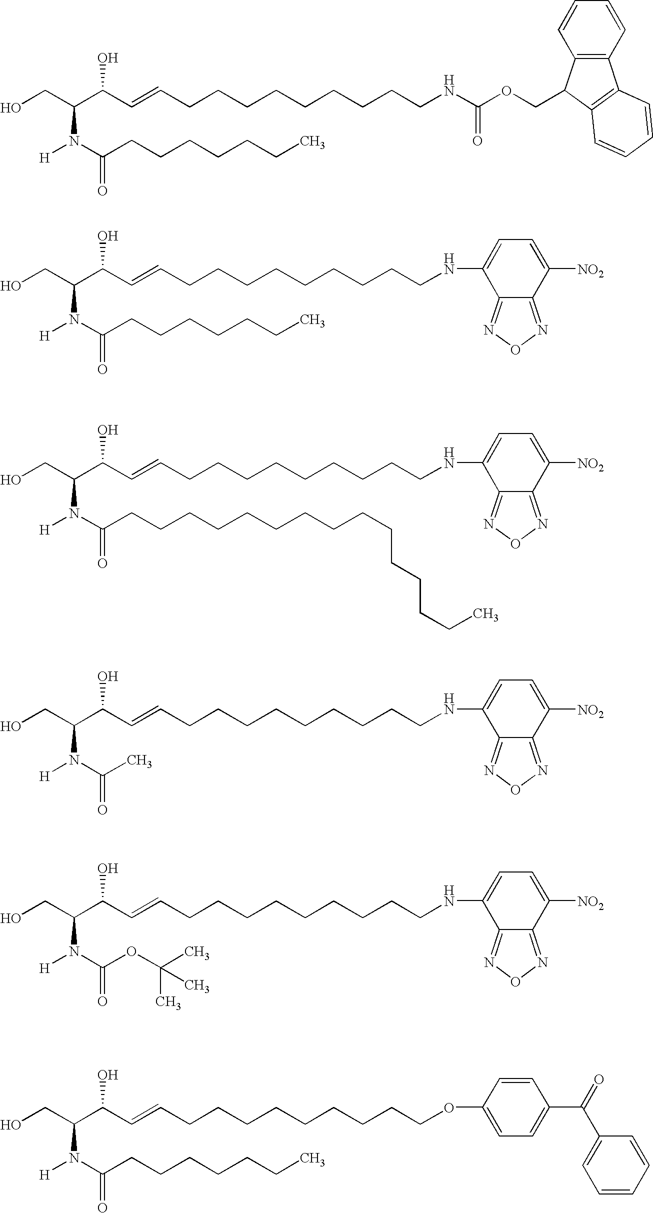

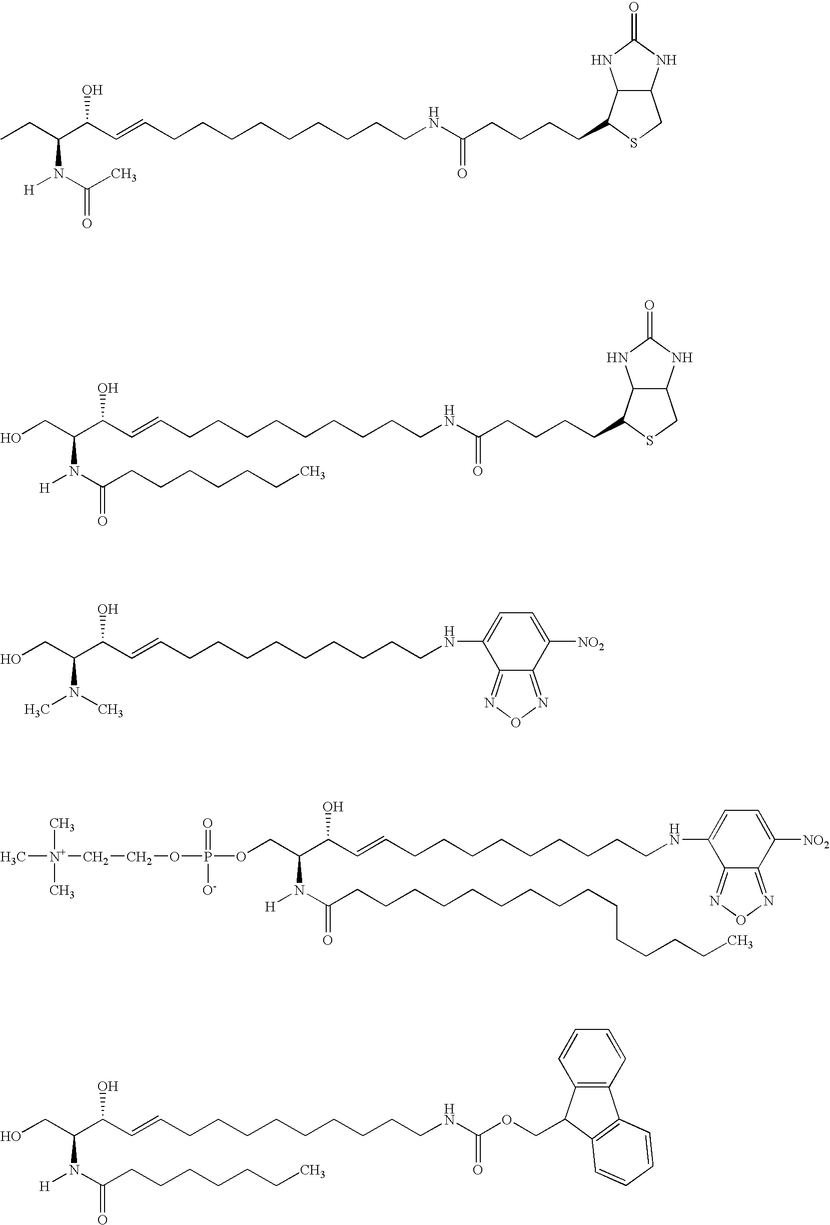

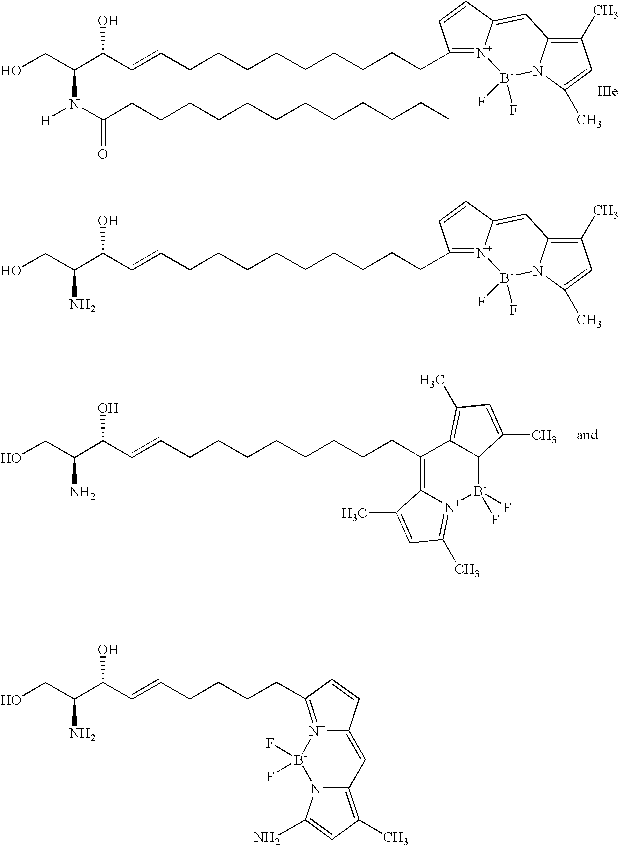

8. A compound according to claim 7 in the form of a salt.

9-10. (canceled)
